(19) United States
(12) Patent Application Publication
(10) Pub. No.: US 2013/0303913 A1
(43) Pub. Date: Nov. 14, 2013

(54) THREE-DIMENSIONAL ULTRASONIC IMAGING METHODS AND SYSTEMS

(71) Applicant: Shenzhen Mindray Bio-Medical Electronics Co., Ltd., Shenzhen (CN)

(72) Inventors: Yong Tian, Shenzhen (CN); Bin Yao, Shenzhen (CN); Yong Jiang, Shenzhen (CN)

(21) Appl. No.: 13/942,462

(22) Filed: Jul. 15, 2013

Related U.S. Application Data
(63) Continuation of application No. PCT/CN2011/084258, filed on Dec. 20, 2011.

(30) Foreign Application Priority Data
Jan. 14, 2011 (CN) 201110008192.7

(51) Int. Cl. A61B 8/08

(52) U.S. Cl. CPC A61B 8/483
USPC 600/447

(57) ABSTRACT

A method for three-dimensional ultrasonic imaging conducting a first group of line scans on a scanning object at a first group of scanning positions; receiving echo signals from the first group of line scans, and acquiring a first group of scanning line data; carrying out a second group of line scans on the scanning object at a second group of scanning positions; receiving echo signals from the second group of line scans and acquiring a second group of scanning line data; and acquiring a three-dimensional image of the scanning object according to scanning data comprising the first group of scanning line data and the second group of scanning line data; the scanning positions in the second group of scanning positions are shifted by a first distance along a direction parallel with a frame scanning direction relative to the scanning position in the first group of scanning positions corresponding to the scanning position, allowing an increase of joint line space without changing the independent line space, thus the imaging quality is improved without reducing the three-dimensional imaging speed.

### TECHNICAL FIELD

The following disclosure relates to methods and systems for three-dimensional ultrasound imaging, and, in particular, to scanning methods in three-dimensional ultrasound imaging and systems using the same.

### BRIEF DESCRIPTION OF THE DRAWINGS

FIG. 1 is a schematic diagrams of line scanning, frame scanning and volume scanning of three-dimensional ultrasound imaging;

FIG. 2 is a schematic diagram of volume data obtained by aligning scanning;

FIG. 3 is a schematic diagram of interlacing volume data obtained by interlacing scanning;

FIG. 4 is a block diagram of a three-dimensional ultrasound imaging system;

FIG. 5 includes schematic diagrams of aligning scanning and interlacing scanning;

FIG. 6 is a schematic diagram of interpolation of interlacing volume data according to an embodiment of the present disclosure;

FIG. 7 is a three-dimensional image obtained by aligning scanning of a three-dimensional image obtained by interlacing scanning according to an embodiment of the present disclosure.

### DETAILED DESCRIPTION

Conventional medical imaging devices can only provide two-dimensional images of the human body. As a result, the sizes and shapes of lesions can only be estimated by doctors based on two-dimensional images. The three-dimensional geometry of a lesion and its surrounding tissues must be imagined by the doctor, leading to difficulties in diagnosis. With the application of three-dimensional visualization technology in ultrasound imaging systems, a three-dimensional image may be reconstructed based on a series of two-dimensional images and then displayed on a monitor. Not only can the overall visual construction of the scanned object (referred to as “object” herein) be obtained from the three-dimensional image, but a significant amount of three-dimensional information may also be saved. Accordingly, three-dimensional ultrasound imaging has been widely used in recent years because it is non-invasive and radiationless, as well as highly flexible for clinical practice.

Three-dimensional ultrasound imaging comprises three steps: acquiring, reconstructing and rendering. Acquiring is the process of obtaining three-dimensional ultrasound volume data. Reconstructing is the process of converting that data into data within a rectangular coordinate system, to obtain volume data whose relative positions are in accordance with those in real space. Thus it is possible to obtain accurate images without deformation. Rendering involves processing the volume data using visualization algorithms to obtain visual information and displaying it on a displaying device.

It can be seen that volume data is the basis for three-dimensional ultrasound images. Therefore, improving the quality of volume data will improve the quality of the images. Since the volume data is comprised of frame data, to improve the quality of the volume data, the quality of frame data must be improved. And since frame data is comprised of line data, frame scanning density must also be improved. The higher the frame scanning density, the better the quality of the frame data.

The frame scanning density can be represented by a reciprocal value of line space. The line space is the distance between adjacent line data within a frame data. Conventionally, to improve the quality of three-dimensional ultrasound images, the line space is reduced to increase the frame scanning density. Thus, when the frame scanning density is increased to N times, the number of line data within a frame data also need to be increased to N times of the original number, and therefore frame scanning time and volume scanning time also need to be increased to N times of the original. Therefore, the imaging speed of three-dimensional ultrasound imaging is reduced to 1/N of the original. However, in a three-dimensional ultrasound imaging system, imaging speed is just as important as image quality. Conventionally, image quality is obtained at the price of reduced speed, and it is impossible to obtain high-quality images at high speeds.

The present disclosure provides methods and systems for three-dimensional ultrasound imaging, which can provide high joint frame scanning density while retaining three-dimensional imaging speed, thereby improving three-dimensional imaging quality.

In one embodiment, a method for three-dimensional ultrasound imaging is provided. The method may include scanning an object with a first group of line scanning at a first group of scanning locations, wherein each of the first group of line scanning is performed at each of the first group of scanning locations. The method may also include receiving echo signals from the first group of line scanning to obtain a first group of scanning line data, wherein at each scanning location of the first group of scanning locations, one of the first group of scanning line data is obtained.

The method may further include scanning the object with a second group of line scanning at a second group of scanning locations, wherein each of the second group of line scanning is performed at each of the second group of scanning locations.

In one embodiment, the method includes receiving echo signals from the second group of line scanning to obtain a second group of scanning line data, wherein at each scanning location of the second group of scanning locations, one of the second group of scanning line data is obtained.

The method may also include forming a three-dimensional image of the object based on a scanning data which comprises at least the first group of scanning line data and the second group of scanning line data; wherein at least one scanning location of the second group of scanning locations is offset by a first distance along a direction parallel to a frame scanning direction relative to a scanning location of the first group of scanning locations which corresponds to said at least one scanning location of the second group of scanning locations.

Embodiments disclosed herein may also include a system for three-dimensional ultrasound imaging. The system may include a probe, a scanning module, and an imaging module. The scanning module may include a drive controlling unit, a scanning controlling, unit and a beam forming and signal processing unit.

In one embodiment, the drive controlling unit and the scanning controlling unit are configured to control the probe to scan an object with a first group of line scanning at a first group of scanning locations, wherein each of the first group of line scanning is performed at each of the first group of scanning locations.

The beam forming and signal processing unit may be configured to receive echo signals from the first group of line scanning to obtain a first group of scanning line data, wherein at each scanning location of the first group of scanning locations, one of the first group of scanning line data is obtained.

In one embodiment, the drive controlling unit and the scanning controlling unit are configured to scan the object with a second group of line scanning at a second group of scanning locations, wherein each of the second group of line scanning is performed at each of the second group of scanning locations.

The beam forming and signal processing unit may be configured to receive echo signals from the second group of line scanning to obtain a second group of scanning line data, wherein at each scanning location of the second group of scanning locations, one of the second group of scanning line data is obtained.

The imaging unit may be configured to form a three-dimensional image of the object based on a scanning data which comprises at least the first group of scanning line data and the second group of scanning line data; wherein at least one scanning location of the second group of scanning locations is offset by a first distance along a direction parallel to a frame scanning direction relative to a scanning location of the first group of scanning locations which corresponds to said at least one scanning location of the second group of scanning locations.

In some embodiments, the second group of scanning locations is offset relative to the first group of scanning locations, which causes the joint line space to be increased while the remaining independent line space is unchanged and causes the joint frame scanning density to be increased without reducing independent frame scanning density, thereby enhancing three-dimensional imaging quality without reducing three-dimensional imaging speed.

Each of the three steps of acquiring, reconstructing, and rendering of three-dimensional ultrasound imaging may be embodied as a module in a system, which may be implemented using any suitable combination of hardware, software, and/or firmware. For example, the system may include a processor and a memory for storing instructions to be executed by the processor. A basic function of an acquiring module is scanning the object, so it may also be referred to as the scanning module. The reconstructing and rendering together constitute the imaging process, so they may also be referred to as the imaging module. Therefore, in one embodiment, a three-dimensional ultrasound imaging system may comprise two modules: a scanning module and an imaging module.

A method performed by the scanning module may include controlling a transducer of a probe to transmit ultrasound waves and receive the echoes at a certain location to obtain a plurality of point data, which are sequentially arranged along the up-down direction of the probe and form one line data (referred to as one line scanning), wherein the direction along which the plurality of point data are arranged (that is, the up-down direction of the probe, or the depth direction of the scanning object or direction of the scanning line) is referred to as the line scanning direction.

The method may further include controlling the location at which the probe transmits ultrasound waves and receives the echoes to be moved along the right-left direction of the probe and performs a plurality of line scanning to obtain a plurality of line data, which are sequentially arranged along the right-left direction of the probe and form one frame data (referred to as one frame scanning), wherein the direction along which the plurality of line data are arranged (that is, the aforementioned right-left direction of the probe, or the direction which is parallel to the frame formed by the line data and perpendicular to the aforementioned line scanning direction) is referred to as the frame scanning direction.

The method may further include controlling the location at which the frame scanning is performed to be moved along the front-back direction and perform a plurality of frame scanning to obtain a plurality of frame data, which are sequentially arranged along the front-back direction of the probe and form one volume data (referred to as one volume scanning), wherein the direction along which said location at which the frame scanning is performed is moved is referred to as the volume scanning direction. This is one complete scanning process of three-dimensional ultrasound imaging.

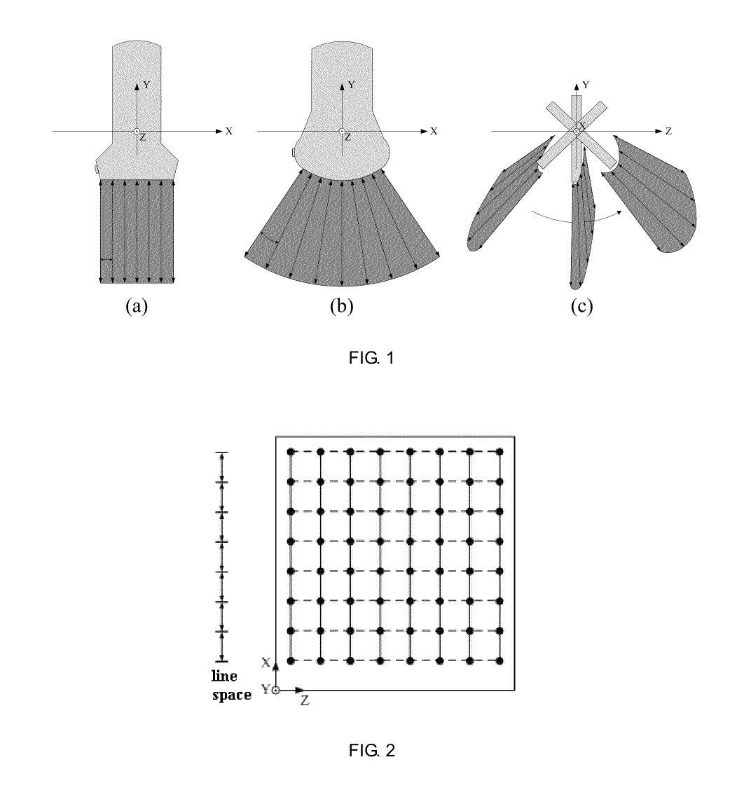

As shown in FIG. 1, FIGS. 1(a) and 1(b) show schematic diagrams of frame scanning of a linear array probe and a convex array probe, respectively, and FIG. 1(c) shows a schematic diagram of volume scanning by fan scanning mode with a convex array probe. In FIG. 1, the X direction is the frame scanning direction, wherein right is positive; the Y direction is the line scanning direction, wherein up is positive; the Z direction is the volume scanning direction, wherein front is positive. Of course, it should be understood by those of skill in the art that the aforementioned positive directions of the respective scanning directions may be defined in other ways as needed.

When describing a physical coordinate of the probe, a representation of the scanning scheme is shown in FIG. 2. The value of X is length for the linear array probe and is angle for the convex array probe; the value of Y is depth of the object; and the value of Z is length for plain scanning and is angle for fan scanning. In FIG. 2, each point represents one line scanning, wherein the direction of the acoustic beam, i.e., the direction along which the data points within the line data are arranged (that is, the aforementioned line scanning direction), is perpendicular to the paper; each of the longitudinal solid lines formed by connecting a plurality of points represents one frame scanning; and the group of all the longitudinal solid lines represents a volume scanning. When numbering the line scanning within each frame scanning in sequence and connecting the line scanning having the same number within the frame scanning, dashed lines as shown in FIG. 2 will be obtained. It can be seen that each of the dashed lines in FIG. 2 is a horizontal straight line, which means that the frame scannings are aligned with each other. In this disclosure, the volume scanning with such an aligning frame scanning is referred to as “aligning scanning.”

As mentioned above, frame scanning density may be represented by the reciprocal value of line space, and the line space is the distance between adjacent line data within a frame data. For a linear array probe, the distance between adjacent line data may be represented by length, as shown by the short line segment in FIG. 1(a). For a convex array probe, the distance between adjacent line data may be represented by angle, as shown by the short arc segment in FIG. 1(b). When being described with the physical coordinate of the probe, line space may be shown by the distance mark on the left of FIG. 2 for both the convex array probe and the linear array probe.

For the aligning scanning shown in FIG. 2, the line space needs to be reduced in order to improve frame scanning density. That is, each frame scanning needs to contain more line scannings. Therefore, the volume scanning will take more time, and the three-dimensional imaging speed will be reduced. According to embodiments of the present disclosure, methods and systems for three-dimensional ultrasound imaging using “interlacing scanning” are provided, which may improve the frame scanning density without reducing the three-dimensional imaging speed, thereby improving the three-dimensional imaging quality.

FIG. 4 is a block diagram of a three-dimensional ultrasound imaging system according to an embodiment of the present disclosure. The system includes a probe, a scanning module, an imaging module, and a displaying device, wherein the scanning module comprises a scanning controlling unit, a drive controlling unit, and a beam forming and signal processing unit, and the imaging module comprises a reconstructing unit and a rendering unit.

In one embodiment, the drive controlling unit generates drive controlling signals which control a transducer to swing in a predetermined way; at the same time the scanning controlling unit generates scanning controlling signals which control the transducer to scan in a predetermined way. Here, “scan” means emitting ultrasound waves and receiving echoes in sequence at a set of locations. That is, a group of pulses, which have been focused with time delays, are sent to the transducer, and then the transducer emits ultrasound waves to the object, receives ultrasound echoes reflected from the object after time delays and converts them to echo signals. The echo signals are transmitted to the beam forming and signal processing unit in which time delay focusing, channel summing and signal processing are performed to obtain an original volume data.

The drive controlling unit and the scanning controlling unit control the transducer to scan to obtain a series of two-dimension ultrasound images between which the spatial relationship can be determined, thereby obtaining real-time three-dimensional original volume data. The original volume data acquired includes of voxels arranged sequentially, each of which represents a point at a certain location within the three-dimensional space being scanned. The spatial relationship may be determined by a plurality of parameters of the acquiring process, including scanning mode (plain scanning, fan scanning), type of probe used (convex array probe, linear array probe, etc.), physical parameters of the probe, region of interest (ROI), motion amplitudes of transducer during volume scanning, etc. In the present disclosure, these parameters are referred to as “acquiring locating parameters.” For an ultrasound imaging system, these acquiring locating parameters are typically known before a three-dimensional ultrasound imaging process starts.

The original volume data and the acquiring locating parameters are sent to the reconstructing unit, in which they are reconstructed to obtain reconstructed volume data. The reconstructed volume data are sent to the rendering unit in which the reconstructed volume data are rendered to obtain visual information such as three-dimensional ultrasound images. Then the visual information is sent to the displaying device to be displayed.

In one embodiment, the scanning controlling unit and the drive controlling unit control the probe to scan in an “interlacing scanning” method. The interlacing scanning method may be described as follows.

The drive controlling unit and the scanning controlling unit may control the probe to scan an object with a first group of line scanning at a first group of scanning locations (that is, a first frame scanning is performed), wherein each of the first group of line scanning is performed at each of the first group of scanning locations.

The beam forming and signal processing unit may receive the echo signals from the first group of line scanning to obtain a first group of scanning line data (i.e., first frame data), wherein at each scanning location of the first group of scanning locations, one of the first group of scanning line data is correspondingly obtained from the echo signals at this scanning location.

In one embodiment, the drive controlling unit and the scanning controlling unit control the probe to scan the scanning object with a second group of line scanning at a second group of scanning locations (that is, a second frame scanning is performed), wherein each of the second group of line scanning is performed at each of the second group of scanning locations.

The beam forming and signal processing unit may receive the echo signals of the second group of line scanning to obtain a second group of scanning line data (i.e., second frame data), wherein at each scanning location of the second group of scanning locations, one of the second group of scanning line data is correspondingly obtained from the echo signals at this scanning location.

In one embodiment, the imaging unit forms a three-dimensional image of the object based on a scanning data which comprises at least the first group of scanning line data and the second group of scanning line data, wherein at least one scanning location of the second group of scanning locations is offset by a first distance along a direction parallel to the frame scanning direction relative to a scanning location of the first group of scanning locations which corresponds to said at least one scanning location of the second group of scanning locations.

In another embodiment, the interlacing scanning method may be performed as follows. The drive controlling unit and the scanning controlling unit may control the probe to scan the object with a third group of line scanning at a third group of scanning locations (that is, a third frame scanning is performed), wherein each of the third group of line scanning is performed at each of the third group of scanning locations.

The beam forming and signal processing unit may receive the echo signals from the third group of line scanning to obtain a third group of scanning line data (i.e., third frame data), wherein at each scanning location of the third group of scanning locations, one of the third group of scanning line data is correspondingly obtained from the echo signals at this scanning location.

In one embodiment, the imaging unit forms a three-dimensional image of the scanning object based on a scanning data which comprises at least the first group of scanning line data, the second group of scanning line data and the third group of scanning line data, wherein at least one scanning location of the third group of scanning locations is offset by a second distance along a direction parallel to the frame scanning direction relative to a scanning location of the first group of scanning locations which corresponds to said at least one scanning location of the third group of scanning locations.

In yet another embodiment, the interlacing scanning method may be performed as follows. The drive controlling unit and the scanning controlling unit may control the probe to scan the scanning object with a fourth group of line scanning at a fourth group of scanning locations (that is, a fourth frame scanning is performed), wherein each of the fourth group of line scanning is performed at each of the fourth group of scanning locations.

The beam forming and signal processing unit may receive the echo signals of the fourth group of line scanning to obtain a fourth group of scanning line data (i.e., fourth frame data), wherein at each scanning location of the fourth group of scanning locations, one of the fourth group of scanning line data is correspondingly obtained from the echo signals at this scanning location.

In one embodiment, the imaging unit forms a three-dimensional image of the scanning object based on scanning data which comprise at least the first group of scanning line data, the second group of scanning line data, the third group of scanning line data, and the fourth group of scanning line data, wherein at least one scanning location of the fourth group of scanning locations is offset by a third distance along a direction parallel to the frame scanning direction relative to a scanning location of the first group of scanning locations which corresponds to said at least one scanning location of the fourth group of scanning locations.

Similarly, in other embodiments, the interlacing scanning method may further comprise similar fifth frame scanning, similar sixth frame scanning, . . . , similar M^{th} frame scanning. Among the scanning locations of every frame scanning, there is at least one scanning location which is offset by a certain distance along a direction parallel to the frame scanning direction relative to a scanning location of the first group of scanning locations which corresponds to said at least one scanning location.

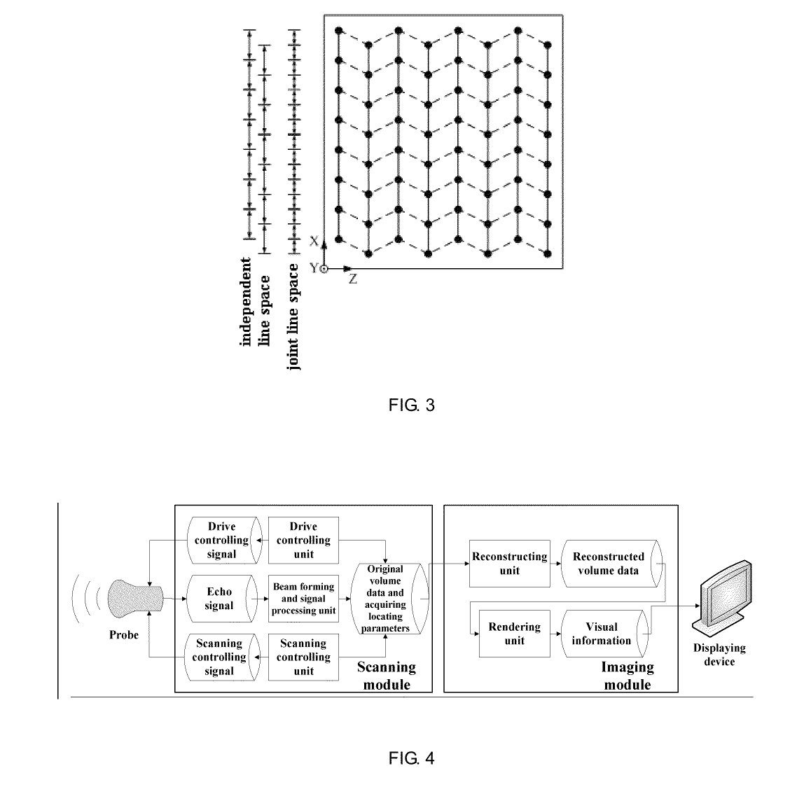

FIG. 3 is a schematic diagram of an implementation of interlacing scanning according to an embodiment of the present disclosure. In FIG. 3, the first column on the left is the first frame data, in which each point represents one scanning line data and each scanning line data corresponds to one scanning location. The second column on the left is the second frame data, in which each point also represents one scanning line data and each scanning line data also corresponds to one scanning location. It can be seen from FIG. 3 that each scanning location of the second frame data is offset by a certain distance along a direction parallel to the frame scanning direction (i.e., the direction of the X-axis in FIG. 3) relative to the corresponding scanning location of the first frame data (for example, the scanning location being connected thereto by a dashed line). In one embodiment, the distance may be half of the line space of the first frame data and/or the second frame data. Of course, the distance may also be any other suitable value.

Similarly, in the embodiment shown in FIG. 3, the fourth frame is offset by a certain distance relative to the third frame, the sixth frame is offset by a certain distance relative to the fifth frame, and so on. It can be seen that in the embodiment shown in FIG. 3 the locations of odd frames remain unchanged while the locations of the even frames are wholly displaced downward by a certain distance (for example, a distance of half of the line space) compared with the aligning scanning in FIG. 2. When numbering the line scanning of every frame scanning in sequence and connecting the line scannings of these frame scannings which have the same number, the connection may be shown by the dashed lines in FIG. 3. It can be seen that every dashed line in FIG. 3 is a sawtooth-shaped polyline, which means that the scanning locations of these frames are interlaced based on odd frames and even frames.

To understand the interlacing scanning scheme, several concepts related to line space and scanning density are introduced. The nature of line space is the distance of line data in X direction. When the frame scanning is considered independently, the distributing distances of the line data of the frames in X direction are referred to as “independent line space,” and the frame scanning density determined by the independent line space is referred to as “independent frame scanning density.” When the volume scanning is considered as a whole, the distributing distance of all the line data in X direction may also be considered as a kind of line space, which is referred to as “joint line space” in the present disclosure, and the frame scanning density determined by the joint line space is referred to as “joint frame scanning density.”

For the aligning scanning scheme in FIG. 2, the joint line space and the independent line space are equal with each other, and therefore the joint frame scanning density and the independent frame scanning density are also equal with each other. For the interlacing scanning scheme in FIG. 3, the independent line space is unchanged, as shown by the leftmost two columns of distance marks in FIG. 3, and therefore the independent frame scanning density is unchanged. But the joint line space is changed to half of the original, as shown by the third column of distance marks on the left in FIG. 3, and therefore the joint frame scanning density is increased to twice that of the original. It has been proofed by experiments according to embodiments of the present disclosure that in a normal controlling range, volume scanning time is usually determined by independent frame scanning density while volume scanning quality is determined by joint frame scanning density. Therefore, for the interlacing scanning scheme according to embodiments of the present disclosure, the independent frame scanning density is unchanged, so the volume scanning time is unchanged. When the joint frame scanning density is increased, the volume scanning quality is improved. Therefore, in embodiments present disclosure, the quality of scanning is improved without decreasing the scanning time.

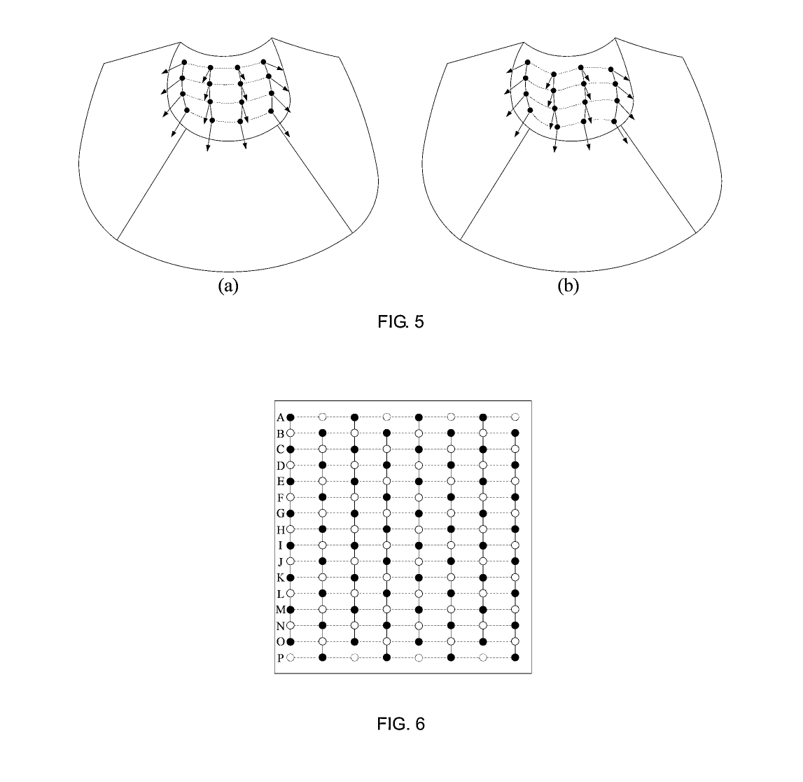

FIG. 2 and FIG. 3 are schematic diagrams of the aligning scanning scheme and the interlacing scanning scheme, respectively, according to embodiments of the present disclosure shown in physical coordinates of the probe. For clarity, their schematic diagrams in real space are shown in FIG. 5. For example, when fan scanning is done with a convex array probe. FIG. 5(a) is a schematic diagram of the aligning scanning scheme, and FIG. 5(b) is a schematic diagram of the interlacing scanning scheme according to embodiments of the present disclosure. For simplicity, only starting points and emitting directions are shown.

The method mentioned above where the odd frames remain unchanged and the even frames are displaced downward by half of the line space may be briefly represented as 0, −0.5 times displacement with a cycle of 2. This is one of the implementations of the interlacing scanning scheme. In other embodiments, an interlacing scanning may be performed in other ways. The frames that are displaced may change: for example, the even frames may remain unchanged and the odd frames may be displaced downward by half of the line space, thereby obtaining a −0.5, 0 times displacement with a cycle of 2; the frames may be displaced upward, for example, the odd frames may remain unchanged and the even frames may be displaced upward by half of the line space, thereby obtaining a 0, 0.5 times displacement with a cycle of 2; the multiple of the displacement may be not 0.5, for example, the odd frames may remain unchanged and the even frames may be displaced upward by one quarter of the line space, thereby obtaining a 0, 0.25 times displacement with a cycle of 2; the cycle may not be 2, for example, the frames whose numbers (for example, the frames may be numbered as follows: in FIG. 2 or FIG. 3, the number of the frames may be started from 1 at the leftmost side and increased in sequence from left to right, as 1, 2, 3, 4, 5, . . . , and so on; of course, the number may be obtained in other ways) have a remainder of 1 when being divided by 3 may remain unchanged, the frames whose numbers have a reminder of 2 when being divided by 3 may be displaced upward by one-third of the line space and the frames whose numbers have a remainder of 0 when being divided by 3 may be displaced upward by two-thirds of the line space, thereby obtaining a 0, ⅓, ⅔ times displacement with a cycle of 3; it is also possible that the frames whose numbers have a remainder of 1 when being divided by 4 remain unchanged, the frames whose numbers have a remainder of 2 when being divided by 4 are displaced downward by one quarter of the line space, the frames whose numbers have a remainder of 3 when being divided by 4 are displaced downward by half of the line space and the frames whose numbers have a remainder of 0 when being divided by 4 are displaced downward by three quarters of the line space, thereby obtaining a 0, 0.25, 0.5, 0.75 times displacement with a cycle of 4; the displacement may even have no cyclicity, for example, a random number sequence may be generated which serves as the multiple of the displacement of the frames, etc. Furthermore, in a continuous three-dimensional ultrasound scanning and imaging process, it is possible to switch between different implementations repeatedly. For example, the odd volume scannings use a 0, −0.5 times displacement with a cycle of 2, and the even volume scannings use a −0.5, 0 times displacement with a cycle of 2, etc.

In embodiments of the present disclosure, the arranging format of the original volume data acquired by interlacing scanning is different from that of aligning scanning. According to the present disclosure, the original volume data acquired by interlacing scanning is referred to herein as “interlacing volume data.” In the reconstructing unit, the interlacing volume data are processed using an “interlacing volume data interpolation” method to obtain real-time three-dimensional ultrasound images. This method is described in detail below.

The interlacing volume data acquired by interlacing scanning according to embodiments of the present disclosure may form a relatively irregular shape. To facilitate reconstructing of volume data, the interlacing volume data may be interpolated to form volume data in a regular cuboid shape.

For example, in one embodiment, the beam forming and signal processing unit may perform the interpolation using at least two scanning line data of the first group of scanning line data and/or the second group of scanning line data aforementioned to obtain interpolating line data.

The interpolating line data obtained by interpolation may be used in reconstructing, rendering, etc., as a part of the first group of scanning line data or the second group of scanning line data or directly as a part of the volume data to form three-dimensional images of the object. That is, the imaging unit forms a three-dimensional image of the object based on the scanning data which comprises at least the first group of scanning line data, the second group of scanning line data and the interpolating line data.

In another embodiment, the beam forming and signal processing unit may perform the interpolation using at least two scanning line data of the first group of scanning line data, the second group of scanning line data and/or the third group of scanning line data aforementioned to obtain interpolating line data.

The imaging unit forms a three-dimensional image of the scanning object based on the scanning data which comprise at least the first group of scanning line data, the second group of scanning line data, the third group of scanning line data and the interpolating line data.

In another embodiment, the beam forming and signal processing unit may perform the interpolation using at least two scanning line data of the first group of scanning line data, the second group of scanning line data, the third group of scanning line data and/or the fourth group of scanning line data aforementioned to obtain interpolating line data.

The imaging unit forms a three-dimensional image of the object based on the scanning data which comprises at least the first group of scanning line data, the second group of scanning line data, the third group of scanning line data, the fourth group of scanning line data and the interpolating line data.

Again, taking the interlacing volume data acquired by the scanning method in FIG. 3 as an example, it is shown in FIG. 6, in which the black points indicate the line data acquired by actual scanning which are referred to as “scanning line data” in this disclosure, and the white points indicate the line data obtained by interpolation which are referred to as “interpolating line data.” The method for interpolation may be averaging two scanning line data which are adjacent to each other in an up-down direction to obtain a mean value and then assigning the mean value to the interpolating line data between them. For example, to obtain the interpolating line data B in FIG. 6, the scanning line data A and the scanning line data C on both sides of B may be used to obtain the interpolating line data B by interpolation. Assuming that the scanning line data A comprises S point data, the values of which are A1, A2, . . . , AS, and the scanning line data C comprises S point data, the values of which are C1, C2, . . . , CS, then the interpolating line data B obtained therefrom also comprises S point data, the values of which may be calculated using a formula: Bi=(Ai+Ci)/2, i=1, 2, . . . , S.

The method mentioned above is suitable for the interpolating line data which do not lie at the edge. The interpolating line data lying at the up edge or low edge (indicated by white points with a dotted line) may be obtained using two scanning line data nearest them through extrapolation assignment. For example, to obtain the interpolating line data P in FIG. 6, the scanning line data O and the scanning line data M may be used to obtain the interpolating line data P by interpolation. Assuming that the scanning line data O and the scanning line data M comprise S point data, respectively, then the value of the point data of the interpolating line data P may be Pi=(3Oi−Mi)/2, wherein Oi is the value of point data of the scanning line data O, Mi is the value of point data of the scanning line data M, and i=1, 2, . . . , S.

In other embodiments, different methods of interpolation may also be used. For example, the interpolating line data which do not lie at the edge may be obtained by spline interpolation using four scanning line data which are adjacent in an up-down direction instead of by linear interpolation using two scanning line data which are adjacent in an up-down direction, or be obtained by linear interpolation using two scanning line data which are adjacent in a right-left direction, or be obtained by spline interpolation using four scanning line data which are adjacent in a right-left direction, or be obtained by any interpolation using a plurality of scanning line data which are adjacent in an up-down and/or right-left direction, etc. The interpolating line data which lie at the edge may be obtained by assigning the value of the respective nearest scanning line data to them, or be obtained by linear interpolation, spline interpolation or any other interpolation in a right-left and/or up-down direction as described above. Or the entire row of line data including scanning line data and interpolating line data which lie at an edge may even be abandoned, etc. The interpolating line data may be located at the original location of the offset scanning line data, that is, at the location of the white points in FIG. 6, or may be located at other locations. In embodiments of the present disclosure, specific methods for interpolating the interlacing volume data and locations of the interpolating line data may be chosen reasonably based on actual conditions.

Interpolating the interlacing volume data in the embodiments above is a pre-processing step before reconstructing volume data. In other embodiments, interpolating the interlacing volume data may not be performed and the reconstructing is directly based on the interlacing volume data.

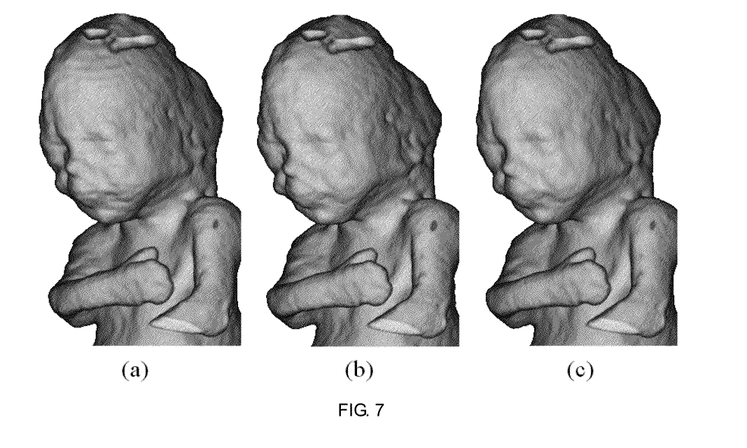

FIG. 7 shows three-dimensional images obtained using the aligning scanning method and a three-dimensional image obtained using the interlacing scanning method. FIG. 7(a) is a resultant image of the aligning scanning method with an independent line space of 1.5 degrees; FIG. 7(b) is a resultant image of the aligning scanning method with an independent line space of 0.9 degree; and FIG. 7(c) is a resultant image of the interlacing scanning method according to embodiments of the present disclosure with an independent line space of 1.5 degrees. It can be seen that the quality of the image in FIG. 7(a) is seriously affected due to very low scanning density, for example, clear transverse stripes are obviously presented in the image. To solve this problem, the scanning density is increased to 1.67 times the original, resulting in the image of FIG. 7(b). Although the quality of the image is greatly improved relative to FIG. 7(a), the imaging speed is reduced to 0.6 times the original. The image in FIG. 7(c) is obtained using the interlacing scanning methods according to embodiments of the present disclosure, which have an imaging speed that is the same as that of FIG. 7(a), and the quality of the image is comparable to FIG. 7(b). Therefore, the interlacing scanning methods according to embodiments of the present disclosure substantially improve the quality of three-dimensional ultrasound images without reducing the imaging speed.

The methods and systems for three-dimensional ultrasound imaging according to embodiments of the present disclosure may be embodied in an ultrasound imaging system by hardware, software, firmware or a combination thereof. Such implementation understood by those of skill in the art.

Although the present disclosure has been described through specific embodiments, present disclosure is not limited to these specific embodiments. Those of skill in the art should understand that various modifications, alternatives, and variations may be made based on the present disclosure, which should be in the scope of protection of the present disclosure. Furthermore, “a (an) embodiment” or “another embodiment” mentioned above may represent different embodiments, or may also be combined completely or partly in one embodiment.

What is claimed is:

1. A method for three-dimensional ultrasound imaging, the method comprising:
scanning an object with a first group of line scanning at a first group of scanning locations, wherein each of the first group of line scanning is performed at each of the first group of scanning locations;
receiving echo signals from the first group of line scanning to obtain a first group of scanning line data, wherein at each scanning location of the first group of scanning locations, one of the first group of scanning line data is obtained;
scanning the object with a second group of line scanning at a second group of scanning locations, wherein each of the second group of line scanning is performed at each of the second group of scanning locations;
receiving echo signals of the second group of line scanning to obtain a second group of scanning line data, wherein at each scanning location of the second group of scanning locations, one of the second group of scanning line data is obtained;
forming a three-dimensional image of the scanning object based on scanning data which comprise at least the first group of scanning line data and the second group of scanning line data;
wherein at least one scanning location of the second group of scanning locations is offset by a first distance along a direction parallel to a frame scanning direction relative to a scanning location of the first group of scanning locations which corresponds to said at least one scanning location of the second group of scanning locations.

2. The method of claim 1, wherein the first distance is half of a distance between the scanning locations of the first group of scanning locations and/or the second group of scanning locations.

3. The method of claim 1, wherein the first distance is one quarter of a distance between the scanning locations of the first group of scanning locations and/or the second group of scanning locations.

4. The method of claim 1, further comprising:
scanning the object with a third group of line scanning at a third group of scanning locations, wherein each of the third group of line scanning is performed at each of the third group of scanning locations;
receiving echo signals from the third group of line scanning to obtain a third group of scanning line data, wherein at each scanning location of the third group of scanning locations, one of the third group of scanning line data is obtained;
forming a three-dimensional image of the object based on scanning data which comprise at least the first group of scanning line data, the second group of scanning line data and the third group of scanning line data;
wherein at least one scanning location of the third group of scanning locations is offset by a second distance along the direction parallel to the frame scanning direction relative to a scanning location of the first group of scanning locations which corresponds to said at least one scanning location of the third group of scanning locations.

5. The method of claim 4, wherein the first distance is one third of a distance between the scanning locations of the first group of scanning locations, the second group of scanning locations and/or the third group of scanning locations, and the second distance is two thirds of the distance between the scanning locations of the first group of scanning locations, the second group of scanning locations and/or the third group of scanning locations.

6. The method of claim 4, further comprising:
scanning the object with a fourth group of line scanning at a fourth group of scanning locations, wherein each of the fourth group of line scanning is performed at each of the fourth group of scanning locations;
receiving echo signals from the fourth group of line scanning to obtain a fourth group of scanning line data, wherein at each scanning location of the fourth group of scanning locations, one of the fourth group of scanning line data is obtained;
forming a three-dimensional image of the object based on scanning data which comprise at least the first group of scanning line data, the second group of scanning line data, the third group of scanning line data and the fourth group of scanning line data;
wherein at least one scanning location of the fourth group of scanning locations is offset by a third distance along the direction parallel to the frame scanning direction relative to a scanning location of the first group of scanning locations which corresponds to said at least one scanning location of the fourth group of scanning locations.

7. The method of claim 6, wherein the first distance is one quarter of a distance between the scanning locations of the first group of scanning locations, the second group of scanning locations, the third group of scanning locations and/or the fourth group of scanning locations, the second distance is half of the distance between the scanning locations of the first group of scanning locations, the second group of scanning locations, the third group of scanning locations and/or the fourth group of scanning locations, and the third distance is three quarters of the distance between the scanning locations of the first group of scanning locations, the second group of scanning locations, the third group of scanning locations and/or the fourth group of scanning locations.

8. The method of claim 1, further comprising:
performing an interpolation using at least two scanning line data of the first group of scanning line data and/or the second group of scanning line data to obtain an interpolating line data;
forming a three-dimensional image of the object based on scanning data which comprise at least the first group of scanning line data, the second group of scanning line data and the interpolating line data.

9. The method of claim 4, further comprising:
performing an interpolation using at least two scanning line data of the first group of scanning line data, the second group of scanning line data and/or the third group of scanning line data to obtain an interpolating line data;
forming a three-dimensional image of the scanning object based on scanning data which comprise at least the first group of scanning line data, the second group of scanning line data, the third group of scanning line data and the interpolating line data.

10. The method of claim 6, further comprising:
performing an interpolation using at least two scanning line data of the first group of scanning line data, the second group of scanning line data, the third group of scanning line data and/or the fourth group of scanning line data to obtain an interpolating line data;
forming a three-dimensional image of the object based on scanning data which comprise at least the first group of scanning line data, the second group of scanning line data, the third group of scanning line data, the fourth group of scanning line data and the interpolating line data.

11. The method of claim 8, wherein the interpolation is linear interpolation, non-linear interpolation or spline interpolation.

12. The method of claim 8, wherein the interpolating line data are located at an original location of offset scanning line data.

13. A system for three-dimensional ultrasound imaging, comprising a probe, a scanning module and an imaging module, the scanning module including a drive controlling unit, a scanning controlling unit, and a beam forming and signal processing unit, wherein:
the drive controlling unit and the scanning controlling unit are configured to control the probe to scan an object with a first group of line scanning at a first group of scanning locations, wherein each of the first group of line scanning is performed at each of the first group of scanning locations;
the beam forming and signal processing unit is configured to receive echo signals of the first group of line scanning to obtain a first group of scanning line data, wherein at each scanning location of the first group of scanning locations, one of the first group of scanning line data is obtained;
the drive controlling unit and the scanning controlling unit are configured to control the probe to scan the object with a second group of line scanning at a second group of scanning locations, wherein each of the second group of line scanning is performed at each of the second group of scanning locations;
the beam forming and signal processing unit is configured to receive echo signals from the second group of line scanning to obtain a second group of scanning line data, wherein at each scanning location of the second group of scanning locations, one of the second group of scanning line data is obtained;
the imaging unit is configured to form a three-dimensional image of the object based on scanning data which comprise at least the first group of scanning line data and the second group of scanning line data;
wherein at least one scanning location of the second group of scanning locations is offset by a first distance along a direction parallel to a frame scanning direction relative to a scanning location of the first group of scanning locations which corresponds to said at least one scanning location of the second group of scanning locations.

14. The system of claim 13, further comprising:
the drive controlling unit and the scanning controlling unit are configured to control the probe to scan the object with a third group of line scanning at a third group of scanning locations, wherein each of the third group of line scanning is performed at each of the third group of scanning locations;
the beam forming and signal processing unit is configured to receive echo signals from the third group of line scanning to obtain a third group of scanning line data, wherein at each scanning location of the third group of scanning locations, one of the third group of scanning line data is obtained;
the imaging unit is configured to form a three-dimensional image of the scanning object based on scanning data which comprise at least the first group of scanning line data, the second group of scanning line data and the third group of scanning line data;
wherein at least one scanning location of the third group of scanning locations is offset by a second distance along the direction parallel to the frame scanning direction relative to a scanning location of the first group of scanning locations which corresponds to said at least one scanning location of the third group of scanning locations.

15. The system of claim 14, further comprising:
the drive controlling unit and the scanning controlling unit are configured to control the probe to scan the scanning object with a fourth group of line scanning at a fourth group of scanning locations, wherein each of the fourth group of line scanning is performed at each of the fourth group of scanning locations;
the beam forming and signal processing unit is configured to receive echo signals from the fourth group of line scanning to obtain a fourth group of scanning line data, wherein at each scanning location of the fourth group of scanning locations, one of the fourth group of scanning line data is obtained;
the imaging unit is configured to form a three-dimensional image of the scanning object based on scanning data which comprise at least the first group of scanning line data, the second group of scanning line data, the third group of scanning line data and the fourth group of scanning line data;
wherein at least one scanning location of the fourth group of scanning locations is offset by a third distance along the direction parallel to the frame scanning direction relative to a scanning location of the first group of scanning locations which corresponds to said at least one scanning location of the fourth group of scanning locations.

16. The system of claim 13, further comprising:
the beam forming and signal processing unit is configured to perform an interpolation using at least two scanning line data of the first group of scanning line data and/or the second group of scanning line data to obtain an interpolating line data;
the imaging unit is configured to form a three-dimensional image of the scanning object based on scanning data which comprise at least the first group of scanning line data, the second group of scanning line data and the interpolating line data.

17. The system of claim 14, further comprising:
the beam forming and signal processing unit is configured to perform an interpolation using at least two scanning line data of the first group of scanning line data, the second group of scanning line data and/or the third group of scanning line data to obtain an interpolating line data;
the imaging unit is configured to form a three-dimensional image of the object based on scanning data which comprise at least the first group of scanning line data, the second group of scanning line data, the third group of scanning line data and the interpolating line data.

18. The system of claim 15, further comprising:
the beam forming and signal processing unit is configured to perform an interpolation using at least two scanning line data of the first group of scanning line data, the second group of scanning line data, the third group of scanning line data and/or the fourth group of scanning line data to obtain an interpolating line data;
the imaging unit is configured to form a three-dimensional image of the scanning object based on scanning data which comprise at least the first group of scanning line data, the second group of scanning line data, the third group of scanning line data, the fourth group of scanning line data and the interpolating line data.

19. The system of claim 16, wherein the interpolating line data are located at an original location of offset scanning line data.
